(19) United States
(12) Patent Application Publication
(10) Pub. No.: US 2007/0197791 A1
(43) Pub. Date: Aug. 23, 2007

(54) Method for the preparation of dicarboxylic lmides

(75) Inventors: Thomas Landmesser, Zurich (CH); Hans Bonnier, Aarburg (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SIEGFRIED Ltd., Zofingen (CH)

(21) Appl. No.: 11/656,895

(22) Filed: Jan. 22, 2007

Related U.S. Application Data
(63) Continuation-in-part of application No. 11/606,527, filed on Nov. 29, 2006.

(30) Foreign Application Priority Data
Jan. 23, 2006 (EP) 06001336.4

(51) Int. Cl. C07D 207/40

(52) U.S. Cl. 546/243; 548/545

(57) ABSTRACT

The present invention relates to a method for the preparation of a carboxylic imide having the general formula
R¹—(CO)—(NR³)—(CO)—R² (I), wherein a carboxylic anhydride having the general formula
R¹—(CO)—O—(CO)—R² (II)

is reacted with urea or a urea derivative of the form (R³HN)—(CO)—(NR³H) in a solvent. In particular, the method can be used for the preparation of thalidomide.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Dicarboxylic imides form part of many substances used in the pharmaceutical field. One of the best known active agents having a dicarboxylic imide function is thalidomide. It was described in 1954 for the first time. In the beginning, thalidomide was used as a sedative. However, in recent years it has been found that thalidomide as well as its derivatives can be used in the treatment of various diseases such as e.g. leprosy, rheumatoid arthritis, AIDS, Crohn's disease as well as cancer diseases. Thalidomide has an immune-suppressive effect as well as an immuno-modulating effect.

Several routes for the synthesis of thalidomide are known from the literature. For an overview see “Axel Kleemann and Jürgen Engel, Pharmaceutical Substances, Thieme Verlag, Stuttgart, 4th edition”, pages 2005-2007. The most widely used variant uses phthalic anhydride as a starting material which is reacted with glutamic acid to yield N-phthaloyl glutamic acid. This acid is reacted with acetic anhydride to form N-phthaloyl glutamic anhydride. The anhydride is then transformed into thalidomide in the melt under the action of urea. During this reaction the typical problems for reactions with gas evolvement in the melt are encountered, e.g. excessive foaming or inferior solubility of the product mixture and thus more difficult processing of the product.

Therefore, it would be helpful to have a method which enables the synthesis of dicarboxylic imides, particularly of thalidomide and its derivatives, by a route where the reaction is performed in solution and therefore can be controlled more easily. It is an object of the present invention to provide a method for the synthesis of dicarboxylic imides in solution.

This object has been achieved by the method according to the independent claim. Advantageous embodiments are set forth in the dependent claims.

### BRIEF SUMMARY OF THE INVENTION

The present invention relates to a method for the preparation of dicarboxylic imides from the corresponding dicarboxylic anhydrides with urea or urea derivates.

### BRIEF DESCRIPTION OF THE DRAWINGS

NOT APPLICABLE

### DETAILED DESCRIPTION OF THE INVENTION

The inventors of the present invention have surprisingly found that reaction of acid anhydrides with urea in a high-boiling solvent results in the synthesis of dicarboxylic imides. This reaction route thus enables e.g. the synthesis of thalidomide starting from N-phthaloyl glutamic anhydride. The synthesis of thalidomide starting from N-phthaloyl glutamic anhydride using sulfolane (tetrahydrothiophene-1,1-dioxide) as a solvent is presented in scheme 1 as an example.

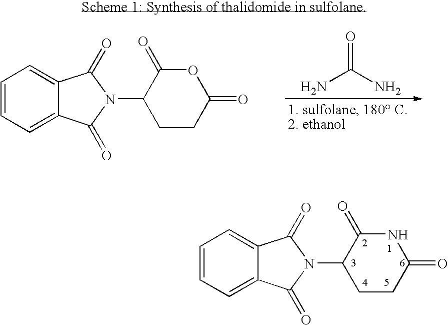

The invention provides a method for the preparation of a dicarboxylic imide having the general formula R¹—(CO)—(NR³)—(CO)—R² (I) wherein a dicarboxylic anhydride of the formula R¹—(CO)—O—(CO)—R² (II) is reacted with urea or a urea derivative having the formula (R³HN)—(CO)—(NR³H) in a solvent to form a dicarboxylic imide (I) wherein R¹, R² and R³ independently of each other can be substituted or unsubstituted, unbranched or branched or cyclic C₁-C₁₀ alkyl, C₂-C₁₀ alkenyl, C₂-C₁₀ alkynyl, C₄-C₁₀ aryl, C₄-C₁₀ heteroaryl, or wherein R¹ and R² can be bound to each other to form a ring, and/or wherein R³ can also be H. If R¹ and R² are bound to each other to form a ring they form together the divalent radical R⁴. Each of the radicals R¹ to R⁴ can be unsubstituted, substituted by one or also by several substituents. An essential feature of the invention is the reaction of the dicarboxylic anhydride with urea or a urea derivative forming the corresponding dicarboxylic imide.

In a preferred embodiment of the invention a method is provided for the preparation of dicarboxylic imides having the general formula (III) 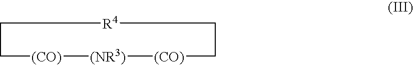
wherein R³ is as defined above, and R⁴ is a divalent radical as defined as R¹ or R², i.e. R⁴ can be a substituted or unsubstituted, unbranched or branched or cyclic C₁-C₁₀ alkanediyl, C₂-C₁₀ alkenylene, C₂-C₁₀ alkynylene, C₄-C₁₀ arylene, C₄-C₁₀ heteroarylene. Preferably, the method is used to prepare substituted or unsubstituted piperidine-2,6-diones wherein R⁴ is substituted or unsubstituted 1,3-propanediyl, particularly preferred substituted or unsubstituted 1 phthalimido-1,3-propanediyl, and in particular 1 phthalimido-1,3-propanediyl for the synthesis of thalidomide.

Whenever any of the residues R¹, R², R³ and/or R⁴ are substituted by a substituent, the substituent may be selected by a person skilled in the art from any known substituent. A person skilled in the art will select a possible substituent according to his knowledge and will be able to select a substituent which will not interfere with other substituents present in the molecule and which will not interfere or disturb possible reactions, especially the reactions described within this application. Possible substituents include without limitation

halogenes, preferably fluorine, chlorine, bromine and iodine;

aliphatic, alicyclic, aromatic or heteroaromatic hydrocarbons, especially alkanes, alkylenes, arylenes, alkylidenes, arylidenes, heteroarylenes and heteroarylidenes;

carbonxylic acids including the salts thereof;

carboxylic acid halides;

aliphatic, alicyclic, aromatic or heteroaromatic carboxylilc acid esters;

aldehydes;

aliphatic, alicyclic, aromatic or heteroaromatic ketones;

alcohols and alcoholates, including a hydroxyl group;

phenoles and phenolates;

aliphatic, alicyclic, aromatic or heteroaromatic ethers;

aliphatic, alicyclic, aromatic or heteroaromatic peroxides;

hydroperoxides;

aliphatic, alicyclic, aromatic or heteroaromatic amides or amidines;

nitriles;

aliphatic, alicyclic, aromatic or heteroaromatic amines;

aliphatic, alicyclic, aromatic or heteroaromatic imines;

aliphatic, alicyclic, aromatic or heteroaromatic sulfides including a thiol group;

sulfonic acids including the salts thereof;

thioles and thiolates;

phosphonic acids including the salts thereof;

phosphinic acids including the salts thereof;

phosphorous acids including the salts thereof;

phosphinous acids including the salts thereof;

The substituents may be bound to the residues R¹, R², R³ and/or R⁴ via a carbon atom, an oxygen atom, a nitrogen atom, a sulfur atom, or a phosphorus atom. The hetero atoms in any structure containing hetero atoms, as e.g. heteroarylenes or heteroaromatics, may preferably N, O, S and P.

In the method according to the invention, high-boiling solvents or solvent mixtures are employed, preferably solvents having a boiling point under atmospheric pressure of more than 150° C., more preferably of more than 170° C., and most preferably of more than 190° C. In this respect, solvents may be selected from aprotic sulfones like e.g. tetrahydrothiophene-1,1-dioxide (sulfolane), saturated lactames like e.g. N-methyl pyrrolidone (NMP), carboxylic amides such like e.g. N,N-dimethyl acetamide (DMA) or formamide, ethers like e.g. diphenyl ether, ureas like e.g. 1,3 dimethyl 2 imidazolidinone (DMI), polyethylene glycols like e.g. diethylene glycol diethylether, aromatics substituted by one or more alkyl groups like e.g. diethylbenzene, pseudocumene, cumene or mesitylene, ionic liquids like e.g. 1-ethyl-3-methyl imidazolium tosylate, siloxanes like e.g. decamethylcyclopentasiloxane, saturated or partially saturated carbocycles like e.g. tetraline or decaline, carbonic esters like e.g. propylene carbonate, and aromatic amines like e.g. N,N-diethylaniline, or the mixtures thereof. Particularly preferred in this respect is tetrahydrothiophene-1,1-dioxide (sulfolane).

| Group | Products |
|---|---|
| aprotic sulfones | tetrahydrothiophene-1,1-dioxide |
| | (sulfolane) |
| saturated lactames | N-methyl pyrrolidone (NMP) |
| carboxylic amids | N,N-dimethyl acetamide (DMA) |
| | formamide |
| ethers | diphenylether |
| ureas | 1,3-dimethyl-2-imidazolidinone (DMI) |
| polyethylene glycols | diethyleneglycol diethylether |
| aromatics substituted by one | diethylbenzene |
| or more alkyl groups | pseudocumene |
| | cumene |
| | mesitylene |
| ionic liquids | 1-ethyl-3-methyl imidazolium tosylate |
| siloxanes | decamethylcyclopentasiloxane |
| saturated or partially saturated | decaline |
| carbocycles | tetraline |
| carbonic esters | propylene carbonate |
| aromatic amines | N,N-diethylaniline |

The method is preferably carried out under atmospheric pressure. However, it is also possible to carry out the method at above or below atmospheric pressure. It is also possible to perform the reaction under a inert gas atmosphere such as nitrogen or argon.

In addition to the educts, foam inhibitors known to those skilled in the art, such as decaline and tetraline, can be used without adversely effecting the reaction.

Subsequent to the reaction, the product may be purified by methods generally known to those skilled in the art. These include for example recrystallization or chromatographic separation. Preferably, the dicarboxylic imide (I) can be purified by recrystallization from an appropriate solvent or solvent mixture. As the solvent for this purpose, methanol, ethanol, dimethylformamide (DMF), water and ethylether, may be used among others. Mixtures of DMF and water, ethylether and methanol, and ethylether and ethanol can be used as the mixtures.

As the reaction is performed in solution, the known problems of reactions in the melt are not encountered. The product can be easily separated from possible contaminations such as side products or remainders of the educts. Dissolution of the solidified melt which has often been difficult can be omitted. The reaction conditions can be easily controlled by the procedures which are well worked out for performing reactions in solution.

In the following the invention will be explained in more detail with respect to Examples without being limited thereto.

Reaction of dicarboxylic anhydrides with urea to form the imides thereof in different solvents

Reactions of phthalic anhydride with urea

### EXAMPLE 1

| | |
|---|---|
| 50 g (0.34 mol) | of phthalic anhydrid were suspended in |
| 75 g | of diphenylether and heated to 175° C. under flushing |
| | with N₂. After the reaction temperature |
| | (175° C.) was reached, |
| 29.2 g (0.49 mol) | of urea was spread in (exothermal). |
| | The reaction mixture was stirred for 30 min at an |
| | internal temperature of 170° C. while N₂ |
| | was constantly supplied. Afterwards, cooling was |
| | performed to an internal temperature of about 90° C. |
| | After this temperature had been achieved |
| 300 g | of ethanol were added quickly. The resulting |
| | suspension was filtered and the filter residue was |
| | washed with ethanol/water (70/30 w/w). Phthalimide |
| | was obtained as a colorless crystalline solid in a |
| | yeild of 68% of the theoretical yield. |

### EXAMPLE 2

In a manner analogue to that of Example 1, a reaction was performed using sulfolane as the solvent. The reaction temperature was 180-185° C. The yield was 66% of the theoretical yield.

### EXAMPLE 3

In a manner analogue to that of Example 1, a reaction was performed using N,N-dimethyl acetamide as the solvent. The reaction temperature was limited to 160° C. The yield was 69% of the theoretical yield.

Reactions of phthaloyl glutamic anhydride with urea

### EXAMPLE 4

| | |
|---|---|
| 50 g (0.193 mol) | of phthaloyl glutamic anhydride were heated |
| | to 180° C. in |
| 75 g | of diethyleneglycol diethylether. |
| | After the reaction temperature was reached |
| 16.5 g (0.275 mol) | of urea were spread in under constant flushing with |
| | N₂ (exothermal). Afterwards, further stirring |
| | was carried out for 1 hour at the reaction |
| | temperature while constant flushing with N2 was |
| | performed. At the end of the reaction period, the |
| | reaction was diluted with dimethylsulfoxide |
| | (DMSO), cooled and then added with ethanol. |
| | Following filtering, washing and drying |
| | 24.9 g (49% of the theoretical yield) |
| | of thalidomide were obtained. |

### EXAMPLE 5

In a manner analogue to that of Example 4, a reaction was performed using pseudocumene as the solvent. The reaction temperature was 160° C. Thalidomide was isolated in a yield of 25%.

### EXAMPLE 6

In a manner analogue to that of Example 4, a reaction was performed using cumene as the solvent. The reaction temperature was 150° C. Thalidomide was isolated in a yield of 11%.

### EXAMPLE 7

In a manner analogue to that of Example 4, a reaction was performed using mesitylene as the solvent. The reaction temperature was 160° C. Thalidomide was isolated in a yield of 23%.

### EXAMPLE 8

In a manner analogue to that of Example 4, a reaction was performed using diethylbenzene as the solvent. The reaction temperature was 170° C. Thalidomide was isolated in a yield of 39%.

### EXAMPLE 9

In a manner analogue to that of Example 4, a reaction was performed using 1-ethyl-3-methyl imidazolium tosylate as the solvent. The reaction temperature was 185° C. Thalidomide was isolated in a yield of 34%.

### EXAMPLE 10

In a manner analogue to that of Example 4, a reaction was performed using decamethylcyclopentasiloxane as the solvent. The reaction temperature was 180° C. Thalidomide could be isolated in a yield of 20%.

### EXAMPLE 11

In a manner analogue to that of Example 4, a reaction was performed using diphenylether as the solvent. The reaction temperature was 185° C. Thalidomide could be isolated in a yield of 38%.

### EXAMPLE 12

In a manner analogue to that of Example 4, a reaction was performed using tetraline as the solvent. The reaction temperature was 180° C. Thalidomide was isolated in a yield of 50%.

### EXAMPLE 13

In a manner analogue to that of Example 4, a reaction was performed using decaline as the solvent. The reaction temperature was 180° C. Thalidomide was isolated in a yield of 48%.

### EXAMPLE 14

| | |
|---|---|
| 50 g (0.193 mol) | of phthaloyl glutamic anhydride were heated |
| | to 180° C. in |
| 75 g | of NMP. |
| | After the reaction temperature was achieved |
| 16.5 g (0.275 mol) | of urea were spread in under constant flushing with |
| | N₂ (exothermal). Afterwards, the stirring was |
| | continued for 1 hour at the reaction temperature |
| | under constant flushing with N₂. At the end of the |
| | reaction period cooling was performed and then |
| | ethanol was added. |
| | Following filtering, washing and drying, |
| | 19.3 g (38% of the theoretical yield) of thalidomide |
| | were obtained. |

### EXAMPLE 15

In a manner analogue to that of Example 14, polyethylene glycol 400 was used as solvent at 185° C. Thalidomide was isolated in a yield of 46%.

### EXAMPLE 16

In a manner analogue to that of Example 14, propylene carbonate was used as solvent at 180° C. Thalidomide could be isolated in a yield of 30%.

### EXAMPLE 17

In a manner analogue to that of Example 14, sulfolane was used as solvent at 180° C. Thalidomide was isolated in a yield of 48%.

### EXAMPLE 18

In a manner analogue to that of Example 14, N,N-diethylaniline was used as solvent at 180° C. Thalidomide was isolated in a yield of 49%.

### EXAMPLE 19

In a manner analogue to that of Example 14, 1,3-dimethyl-2-imidazolidinone (DMI) was used as solvent at 185°. Thalidomide could be isolated in a yield of 40%.

### EXAMPLE 20

In a manner analogue to that of Example 14, formamide was used as solvent at 185° C. Thalidomide could be isolated in a yield of 35%.

### EXAMPLE 21

| | |
|---|---|
| 75 g | of sulfolane were heated to 175° C. |
| | At this temperature, a mixture of |
| 50 g (0.193 mol) | of phthaloyl glutamic anhydride and |
| 16.5 g (0.275 mol) | of urea was spread in under constant flushing |
| | with N₂. Afterwards, the stirring was continued |
| | for approx. 2 hours at about 180° C. under constant |
| | flushing with N₂. At the end of the reaction |
| | period, cooling was performed and then |
| 285 g | ethanol were added. |
| | After filtering, washing and drying |
| | 24.3 g (48% of the theoretical yield) of |
| | thalidomide were obtained. |

Reactions of adipic anhydride with urea

### EXAMPLE 22

| | |
|---|---|
| 20 g (0.156 mol) | of adipic anhydride were heated in |
| 30 g | of sulfolane to a reaction temperature of 180° C. |
| | After the reaction temperature was achieved |
| 13.5 g (0.24 mol) | of urea were spread in and stirring was continued |
| | for 1 h at the reaction temperature under flushing |
| | with N₂. Following cooling, the reaction mixture |
| | was first added with 2-propanol and then with |
| | methyl-tert. butylether (MTBE). Adipic imide was |
| | isolated in a yield of 76%. |

### EXAMPLE 23

In a manner analogue to that of Example 22, diethyleneglycol diethylether was used as solvent at 180° C. Adipic imide was isolated in a yield of 56%.

Reactions of 2-methyl succinic anhydride with urea

### EXAMPLE 24

| | |
|---|---|
| 25 g (0.219 mol) | of 2-methyl succinic anhydride were heated in |
| 37.5 g | of sulfolane to a reaction temperature of 180° C. |
| | After the reaction temperature was achieved |
| 18.95 g (0.32 mol) | of urea were spread in and stirring was continued |
| | for 1 h at the reaction temperature under flushing |
| | with N₂. Following cooling, the reaction mixture |
| | was first added with 2-propanol and then with |
| | MTBE. 6.5 g of 2-methyl succinic imide (32% of |
| | the theoretical yield) were obtained. |

### EXAMPLE 25

In a manner analogous to that of Example 24, diethyleneglycol diethylether was used as the solvent at 180° C. After cooling, first MTBE was added. From the resulting oil 2-methyl succinic imide was obtained in a yield of 20% by recrystallization from ethanol.

What is claimed is:

1. A method for the preparation of a dicarboxylic imide having the general formula (I)
R¹—(CO)—(NR³)—(CO)—R² (I),
wherein a dicarboxylic anhydride having the general formula (II)
R¹—(CO)—O—(CO)—R² (II)
is reacted with urea or a urea derivative of the form (R³HN)—(CO)—(NR³H) in a solvent to form a dicarboxylic imide (I),
wherein
R¹, R² and R³ independently of one another can be substituted or unsubstituted, unbranched or branched or cyclic C₁-C₁₀ alkyl, C₂-C₁₀ alkenyl, C₂-C₁₀ alkynyl, C₄-C₁₀ aryl, C₄-C₁₀ heteroaryl, or wherein R¹ und R² can be bound to each other to form a ring, and/or wherein R³ can also be H.

2. The method according to claim 1 for the preparation of dicarboxylic imides having the general formula (III) 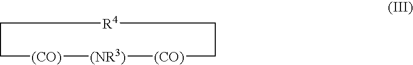
wherein
R³is as defined in claim 1, and R⁴ can be a substituted or unsubstituted, unbranched or branched or cyclic C₁-C₁₀ alkanediyl, C₂-C₁₀ alkenylene, C₂-C₁₀ alkynylene, C₄-C₁₀ arylene, C₄-C₁₀ heteroarylene.

3. The method according to claim 2 for the preparation of substituted or unsubstituted piperidine-2,6-diones wherein R⁴ is a substituted or unsubstituted 1,3-propanediyl.

4. Method according to claim 3 for the preparation of unsubstituted or substituted 3 phthalimidopiperidine-2,6-diones wherein R⁴ is an unsubstituted or a substituted 1 phthalimido-1,3-propanediyl.

5. Method according to claim 1 wherein the solvent is a high-boiling solvent having a boiling point of more than 150° C., preferably of more than 170° C., most preferably of more than 190° C.

6. Method according to claim 1 wherein the solvent is selected from the group consisting of aprotic sulfones, saturated lactames, carboxylic amides, ethers, ureas, polyethylene glycols, aromatics substituted by one or more alkyl groups, ionic liquids, siloxanes, saturated or partially saturated carbocycles, carbonic esters, aromatic amines, or the mixtures thereof.

7. Method according to claim 6, wherein the aprotic sulfone is tetrahydrothiophene-1,1-dioxide (sulfolane), the saturated lactame is N-methyl pyrrolidone (NMP), the carboxylic amide is N,N-dimethyl acetamide (DMA) or formamide, the ether is diphenyl ether, the urea is 1,3 dimethyl 2 imidazolidinone (DMI), the polyethylene glycol is diethyleneglycol diethylether, the aromatic substituted by one or more alkyl groups is selected from diethylbenzene, pseudocumene, cumene or mesitylene, the ionic liquid is 1-ethyl-3-methyl imidazolium tosylate, the siloxane is decamethylcyclopentasiloxane, the saturated or partially saturated carbocycle is tetraline or decaline, the carbonic ester is propylene carbonate, and/or the aromatic amine is N,N-diethylaniline, and wherein tetrahydrothiophene-1,1-dioxide is preferably used as the aprotic sulfone.

8. Method according to claim 1 wherein the temperature during the reaction is in a range of 140° C. to 220° C., preferably in a range of 150° C. to 210° C., even more preferably in a range of 160° C. to 200° C.

9. Method according to claim 1 wherein the substances are reacted under atmospheric pressure.

10. Method according to claim 1 wherein in addition a foam inhibitor is employed.

11. Method according to claim 10 wherein the foam inhibitor is selected from the group consisting of decaline and tetraline.

12. Method according to claim 1 wherein the dicarboxylic imide (I) is purified in a subsequent step by recrystallization or by chromatographic purification procedures.

13. Method according to claim 12 wherein for recrystallization of the dicarboxylic imide (I) a suitable solvent or solvent mixture, preferably a solvent or solvent mixture selected from the group consisting of methanol, ethanol, a mixture of DMF and water, a mixture of ethylether and methanol, and a mixture of ethylether and ethanol is employed.
